(19) United States
(12) Patent Application Publication
(10) Pub. No.: US 2009/0204044 A1
(43) Pub. Date: Aug. 13, 2009

(54) FINGER SPLINT ASSEMBLY AND METHOD OF TREATING MALLET FINGER

(76) Inventor: Catherine Benison, Northborough, MA (US)

Correspondence Address: CESARI AND MCKENNA, LLP, 88 BLACK FALCON AVENUE, BOSTON, MA, 02210, US

(21) Appl. No.: 12/027,646

(22) Filed: Feb. 7, 2008

(51) Int. Cl. A61F 5/01

(52) U.S. Cl. 602/22

(57) ABSTRACT

A finger splint assembly includes a generally cylindrical sleeve having a wall of flexible but inextensible material and opposite proximal and distal ends which are open. The remaining component of the assembly is a shim having opposite relatively thick and relatively thin ends and a flat top surface extending between those ends. The shim is longer than the sleeve such that when the shim is inserted, thin end first, into the distal end of the sleeve with its top surface aligned with an internal chord of the sleeve and with one of the thick and thin ends lying within the sleeve, the other of the thick and thin ends projects from the corresponding end of the sleeve. A method of using the assembly to treat Mallet Finger is also disclosed.

### BACKGROUND OF THE INVENTION

1. Field of the Invention

This invention relates to an orthopedic treatment. It relates especially to a finger splint assembly and method for the non-surgical treatment of Mallet Finger.

2. Background Information

Mallet Finger is the name given to a finger deformity due to damage of the extensor tendon of a finger or thumb. This is the thin tendon that straightens the digit. The result is that the weaker extensor tendon is overcome by the more robust tendons that bend a finger F with the result that the end of the finger F beyond the joint J is not able to be straightened and assumes the position shown in FIG. 1A. Mallet Finger usually occurs when an object strikes the tip of the finger, or vice versa. If the force of the blow is strong enough, it may even pull away some of the finger bone along with the tendon. This condition is a common affliction among baseball and basketball players.

Usually Mallet Finger injuries can be treated without surgery by applying a splint to the finger. The simplest splint S is an elongated rigid plate positioned on top of the finger F so as to bridge the area of finger joint J as shown in FIG. 1B. The opposite ends of the splint S are secured to the finger by adhesive tape T wound around the splint and finger so that the end of the finger is held straight as shown in that figure. Other conventional splints are designed so that they extend both over and under the finger joint J as shown at S′ in FIG. 1C; they are also secured to the finger by adhesive tape T. Splints such as these must remain in place until the extensor tendon heals which can take as long as eight weeks. After that time, patients may gradually wear the splint less frequently until the finger regains its full function and no longer droops as shown in FIG. 1A.

Conventional finger splints such as the ones just described are disadvantaged in that they inhibit bending of the finger at the adjacent finger joint J′ because the tape T which holds the splint in place is too close to that joint. Also, splints which are held in place by adhesive tape must be kept dry because sweat and moisture degrade the adhesive properties of the tape. As a practical matter, however, this is not possible. Invariably, the wearer's hand gets wet, causing the splint to slip from the finger. The result is that conventional splints have to be repeatedly repositioned and retaped. Not only is this an annoyance, it also extends the healing period for the Mallet Finger injury. Moreover, even if the tape does remain in place, after a few weeks it becomes dirty and unsightly and a site for bacterial buildup.

Furthermore, when it does become necessary to remove the prior splints for one reason or the other, this is difficult to do using only one hand.

### SUMMARY OF THE INVENTION

Thus, the present invention aims to provide an improved splint assembly for the treatment of Mallet Finger.

Another object of the invention is to provide such a splint assembly which does not involve the use of adhesive tape.

A further object of the invention is to provide a splint assembly which is unaffected by immersion in water and can be kept clean.

Still another object of the invention is to provide a splint assembly such as this which is quite easy to affix to a finger or thumb following a Mallet Finger injury.

A further object of the invention is to provide a finger splint assembly for the treatment of Mallet Finger which still allows the wearer to bend the finger at the unafflicted finger joint(s).

Yet another object is to provide such a splint assembly which tends to remain in place for a prolonged period, yet which can be removed easily using only one hand.

Another object of the invention is to provide a finger splint assembly which is relatively easy and inexpensive to make.

Yet another objective is to provide a method of treating Mallet Finger which provides one or another of the above advantages.

Other objects will, in part, be obvious and will, in part, appear hereinafter.

The invention accordingly comprises the several steps and the relation of one or two more of such steps with respect to each of the others and the assembly possessing the features, properties and relation of elements, which are exemplified in the following detailed disclosure and the scope of the invention will be indicated in the claims.

All of the above objects are achieved by a finger splint assembly comprised of a generally cylindrical open-ended sleeve having a wall of relatively flexible but relatively inextensible material and which is adapted to be slid onto the end of a finger so as to cover the injured finger joint area thereof. The length of the sleeve should be somewhat less than the length of the finger segment between the tip of the finger and the proximal finger joint beyond the injured one, and the inside diameter of the sleeve should be somewhat greater than the diameter of that segment so that the sleeve can slidably receive that segment.

The remaining component of the assembly is a shim which may be slid into the distal end of the sleeve between the sleeve wall and the underside of the finger. The dimensions of the shim are such that the finger segment is captured between the sleeve wall and the shim, thereby releasably locking the sleeve to the finger.

When the shim is in place, it is located substantially entirely within the sleeve and, together with the sleeve, holds the captured finger segment substantially straight in extension which is the optimum position for treating a Mallet Finger injury. As we shall see, the sleeve is preferably perforated or permeable so that ambient air can reach the captured finger segment. Also, both the sleeve and the shim are made of materials which are unaffected by moisture so that the wearer's hand with the Mallet Finger injury may be immersed in water without adversely affecting the splint assembly. Accordingly, the splint assembly can be worn continuously for the entire time it takes for the injury to heal without causing undue discomfort to the wearer. Yet if it should be necessary to remove the splint assembly, that can be done relatively easily using only one hand.

### BRIEF DESCRIPTION OF THE DRAWINGS

For a fuller understanding of the nature and objects of the invention, reference should be made to the following detailed description taken in connection with the accompanying drawings, in which:

FIG. 1A, already described, is a fragmentary perspective view of a finger with a Mallet Finger injury;

FIGS. 1B and 1C are similar views showing conventional splints for treating the injury depicted in FIG. 1A;

FIG. 2 is an exploded perspective view of a finger splint assembly comprised of a sleeve and a shim according to the invention;

FIG. 3A is an elevational view of the FIG. 2 assembly installed on a finger;

FIG. 3B is a sectional view taken along line 3B-3B of FIG. 3A;

FIG. 4A is a view similar to FIG. 3A illustrating another splint assembly embodiment;

FIG. 4B is a sectional view taken along line 4B-4B of FIG. 4A, and

FIGS. 5A and 5B are the top plane and side elevational views, respectively, of another shim embodiment for use in my splint assembly.

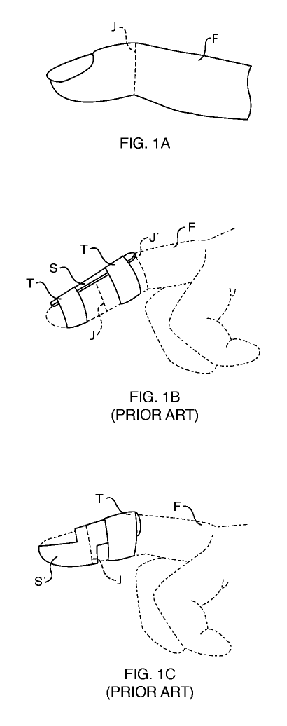

### DETAILED DESCRIPTION OF ILLUSTRATIVE EMBODIMENTS

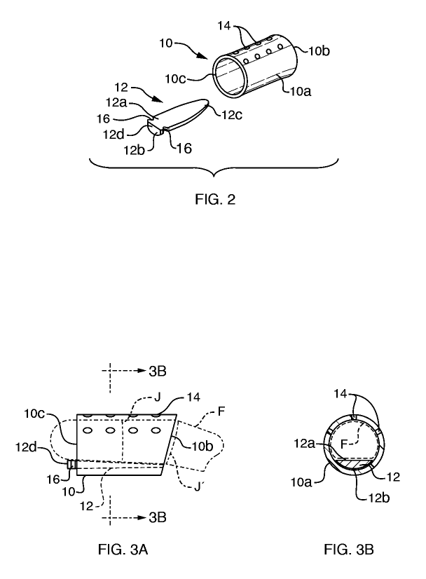

Referring to FIG. 2 of the drawings, my finger splint assembly includes a generally cylindrical sleeve indicated at 10. The sleeve has a side wall 10a, a proximal end 10b and a distal end 10c. Preferably, the ends of the wall are rounded off to avoid sharp edges. The sleeve 10 is of a somewhat flexible but inextensible material so that wall 10a can flex radially but the sleeve as a whole is resistant to bending. Also, the sleeve should be of a material which is unaffected by moisture. A suitable sleeve material is standard vinyl plastic tubing having a wall thickness in the order of at least 1/16 inch.

Perforations 14 may be present in an upper area of the sleeve wall 10a to allow penetration of air into the sleeve from the side. Alternatively, sleeve 10 may be made of a conventional, inextensible, permeable plastic material.

Still referring to FIG. 2, the remaining component of my assembly is a device for releasably securing sleeve 10 to a finger F, to wit: a shim shown generally at 12. The illustrated shim is a wedge-shaped body having a flat top surface 12a and an undersurface 12b that is curved, as best seen in FIG. 3B, to correspond generally to the curvature of the inside surface of the sleeve wall 10a. The shim 12 is tapered in both width and thickness from its proximal end 12c to its distal end 12d so that the shim becomes progressively wider and thicker toward the latter end. Preferably, the thin end 12c is fairly small in the plane of surface 12a to facilitate its insertion into sleeve 10 as will be described. Desirably also, shim 12 has vertical notches 16 in the opposite sides of the shim adjacent to the thicker end 12d to facilitate gripping that end.

The dimensions of shim 12 are coordinated with those of sleeve 10. More particularly, as noted above, the cross-sectional curvatures of the shim undersurface 12b and tube wall 10a are more or less the same. Also, the wedge is somewhat longer than the sleeve so that when the wedge is inserted into the sleeve with one end reposing inside the sleeve at one end thereof, the other end of the wedge projects from the other end of the sleeve.

In accordance with the invention, shim 12, like sleeve 10, is made of a material that is unaffected by water and moisture. Thus it may be of metal, wood or plastic material. I have found that a wood shim is particularly desirable because wood is a forgiving material which is comfortable and warm next to the skin and it has antibacterial properties.

In order to use my splint assembly, the sleeve 10 is slid onto the end of a Mallet Finger F so that the finger joint J with the injured extensor tendon is midway between the ends of the sleeve as shown in FIG. 3. Preferably, the proximal end 10b of the sleeve is cut on a bias so that the sleeve end 10b is spaced far enough from the underside of the adjacent finger joint J′ that it does not affect the bending of the finger at that joint. The distal end of the sleeve may be square cut as shown to expose the entire tip of finger F so that the wearer can still touch things and use that finger to perform useful tasks; e.g., typing on a keyboard.

If the sleeve 10 has perforations 14, they should be located in the upper, longer region of wall 10a so that they do not compromise the stiffness of the shorter sleeve wall portion at the underside of the finger F.

Once the sleeve 10 has been positioned on finger F, the shim 12 may be slid, thin end 12c first, into the distal end 10c of the sleeve underneath finger F as shown in FIGS. 3A and 3B. As the shim is pushed into the sleeve, it wedges against the underside of the finger and the sleeve wall 10a thereby drawing the top and sides of the sleeve wall against the finger so that the finger segments on opposite sides of joint J are captured between the sleeve wall 10a and the upper surface 12a of the shim as best seen in FIG. 3B. The shim is pushed into the sleeve until only the thick end 12d of the shim with notches 16 projects from the distal end of the sleeve. In other words, the thin end 12c of the shim may sit just inside the proximal end of the sleeve. When the shim is so seated, the shim surface 12a is aligned with an interior chord of sleeve 10 and the shim undersurface 12b is flush against the sleeve wall 10a. Thus, the sleeve 10 is firmly fixed to finger F at the joint J so that the captured finger segment is perfectly straight and maintained in extension. Any force tending to slide sleeve 10 from finger F causes the distal end of the sleeve to “ride up” on the shim 12, resulting in a further tightening of the sleeve around the finger which counteracts that force. Thus the assembly can remain in place on finger F for the time it takes for the injured finger to heal.

Since the sleeve wall 10a is somewhat flexible in the radial direction, it can accommodate slight changes in the cross sectional shape of the finger due to temperature and blood pressure changes in the finger without adversely affecting the overall resistance of the sleeve to bending. Also, since the sleeve and shim are made of materials that are unaffected by moisture, the wearer of the splint assembly can still perform duties customarily involving the wetting of the hands without adversely affecting the splint assembly or its ability to treat the Mallet Finger injury.

When it becomes necessary or desirable to remove the splint assembly from the finger F, that can be done easily using only one hand by first withdrawing shim 12 from the sleeve using the notches 16 to grip the exposed end of the shim and then sliding the sleeve 10 from the finger.

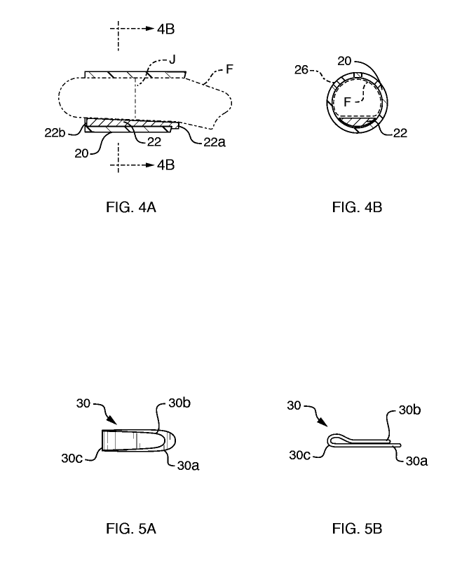

Refer now to FIGS. 4A and 4B, which illustrate a splint assembly comprised of a sleeve 20 made of a permeable plastic material so that air can reach the entire surface of the finger F. This assembly includes a shim 22 having a proximal, thin end 22a which projects beyond the proximal end of the sleeve when the thicker end 22b of the shim is just inside the distal end of the sleeve as shown in FIG. 4A. Preferably, the shim end 22a is stepped or barbed as shown so that its end 22a overhangs the proximal end of the sleeve wall. This prevents the shim from being pushed out of the sleeve when the finger F is bent at the adjacent joint J′. The assembly shown in FIGS. 4A and 4B may be removed from the finger by pushing the shim end 22a up and toward the finger tip which releases the shim from the sleeve and exposes its thicker end 22b. The shim can then be withdrawn from the sleeve and the sleeve slid off the finger. In all other respects, the assembly depicted in FIGS. 4A and 4B has the same advantages discussed above in connection with FIGS. 2 and 3.

FIGS. 5A and 5B show a shim 30 cut or stamped from relatively stiff plastic sheet material which is folded over to create a varied thickness. This shim includes a lower leaf 30a and an upper leaf 30b joined at a relatively thick crease 30c. The two leaves are bonded together face to face to form a structure which is resilient enough to spread apart the sides of sleeve 10 or 20. Thus, when shim 30 is inserted into the sleeve, finger F is captured between the upper portion of the sleeve wall and the shim as described above. Shim 30 is advantaged in that it is quite inexpensive to make in quantity.

The components of my splint assembly can be made in a number of standard sizes to fit different size fingers. This can be done quite economically because vinyl tubing of many diameters is available off the shelf and may be cut easily to different lengths to form the sleeves. Likewise, the shims may be formed in various standard sizes to fit the sleeves. If desired, the two components of the assembly may be color coded to denote a particular finger size that the assembly will fit.

In addition, the sleeve component of my assembly may be customized easily to suit special conditions. For example, a hole or internal recess may be formed in the top of the sleeve wall to provide clearance for an enlarged bone knuckle at the finger joint J as shown at 26 in FIG. 4A.

It is apparent from the foregoing that my splint assembly for treating finger injuries such as Mallet Finger is superior to conventional such splints in that it does not require adhesive tape to secure the splint to the finger. On the contrary, it is made entirely of materials which are impervious to moisture such that the hand with the injured finger can be immersed in water allowing the wearer to perform everyday duties involving the wetting of hands. Thus, my splint assembly can remain on an injured finger continuously until the injury has healed. Also, since the components of the assembly may be made of materials which are somewhat compliant, e.g. vinyl plastic and wood, the assembly does not cause undue discomfort to the wearer. Finally, it is important to note that the components of the assembly can be assembled on and disassembled from an injured finger easily by relatively unskilled medical personnel or even by the injured person using only one hand. Therefore, the assembly should find wide acceptance in the relevant marketplace.

It will thus be seen that the objects set forth above among those made apparent from the preceding description are efficiently attained. Also, since certain changes may be made in carrying out the above method and in the construction set forth without departing from the scope of the invention, it is intended that all matter contained in the above description or shown in the accompanying drawings shall be interpreted as illustrative and not in a limiting sense.

It should also be understood that the following claims are intended to cover all of the generic and specific features of the invention described herein.

1. A finger splint assembly comprising
a generally cylindrical sleeve, said sleeve having a wall of relatively flexible but substantially inextensible material and opposite proximal and distal ends which are open, and
a shim having opposite relatively thick and relatively thin ends and a substantially flat top surface extending between said ends, said shim being longer than the sleeve such that when the shim is inserted, thin end first, into the distal end of the sleeve, with its top surface aligned with an internal chord of the sleeve and with one of said thick and thin ends lying within the sleeve, the other of said thick and thin ends projects from the corresponding end of the sleeve.

2. The assembly defined in claim 1, wherein the proximal end of the sleeve is cut on a bias and the distal end of the sleeve is square cut so that an upper region of the sleeve is longer than a lower region thereof.

3. The assembly defined in claim 2, and further including a plurality of holes extending through said wall in said upper region of the sleeve.

4. The assembly defined in claim 1, wherein said sleeve is of a permeable plastic material.

5. The assembly defined in claim 1, wherein said wall has rounded edges at the opposite ends of the sleeve.

6. The assembly defined in claim 1, wherein said sleeve comprises a length of vinyl tubing.

7. The assembly defined in claim 1, wherein said shim is a solid wedge-shaped body which is wider at said thick end than at said thin end.

8. The assembly defined in claim 7, wherein the body has a cross-sectionally curved undersurface whose curvature substantially matches the cylindrical curvature of the sleeve wall so that when said top surface is aligned with said chord, the undersurface lies flush against the sleeve wall.

9. The assembly defined in claim 7, and further including a barb at the thin end of the shim which engages over the edge of the sleeve wall when the thick end of the shim reposes within said sleeve.

10. The assembly defined in claim 7, and further including gripping means at said thick end of the shim.

11. The assembly defined in claim 10, wherein the gripping means comprise at least one notch in a side of said body.

12. The assembly defined in claim 10, wherein the gripping means comprise an enlargement of said body.

13. The assembly defined in claim 1, wherein the shim comprises a flexible resilient plastic plate which is wider at said thick end than at said thin end.

14. The assembly defined in claim 13, wherein said plate comprises two leaves folded together face to face with a crease at said thick end.

15. The assembly defined in claim 14, wherein the leaf faces are bonded together.

16. A method of treating a Mallet Finger having an injured joint comprising the steps of
forming a generally cylindrical sleeve having a wall of flexible but inextensible material and proximal and distal ends which are open;
sliding the sleeve onto a Mallet Finger whose diameter is slightly smaller than the inside diameter of the sleeve until the sleeve bridges said joint, and
inserting a shim between the underside of the finger and the sleeve wall so as to releasably capture the finger between an upper portion of the sleeve wall and the shim.

17. The method defined in claim 16 including the step of forming at least a portion of the sleeve wall with pores or perforations.

18. The method defined in claim 16 including the step of forming the shim as a solid wedge-shaped body with a flat top surface and an undersurface which is curved to match the cylindrical curvature of the sleeve wall.

19. The method defined in claim 18 including the steps of forming the sleeve of vinyl plastic and the shim of wood.

20. The method defined in claim 19 including the step of cutting the proximal end of the sleeve on a bias.
